(19) United States
(12) Patent Application Publication
(10) Pub. No.: US 2005/0119539 A1
(43) Pub. Date: Jun. 2, 2005

(54) System for analyzing the skin

(75) Inventor: Roland Bazin, Bievres (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 10/963,645

(22) Filed: Oct. 14, 2004

(30) Foreign Application Priority Data
Oct. 16, 2003 (FR) 03 12105

(51) Int. Cl. G06F 17/60; G06K 9/00; A61B 5/00

(52) U.S. Cl. 600/315; 382/128; 705/2

(57) ABSTRACT

The present invention relates to a system for analyzing the skin of a subject, the system comprising: a camera for acquiring at least one image of the skin at a magnification that enables the pores and/or the spots of the skin to be observed; a display device for displaying simultaneously at least one acquired image and an image of a reference skin; an apparatus other than a camera for measuring at least one physico-chemical characteristic of the skin; and a processor for delivering information relating to the state of the skin by processing at least one acquired image and/or a measurement performed by the apparatus.

The present invention relates to a system for analyzing the skin of a human being.

Experience shows that consumers often do not choose the best makeup and/or care products for their skin because they do not always know accurately their own skin type and its specific needs.

Consequently, there exists a need to be able to analyze the skin in a manner that is satisfactory, for example for the purpose of providing consumers with personalized advice.

The invention provides a system for analyzing the skin, the system comprising:
a camera for acquiring at least one image of the skin at a magnification that enables the pores and/or the spots of the skin to be observed;
a display device for displaying simultaneously at least one acquired image and an image of a reference skin, which images may correspond for example to the same portion of the human being;
an apparatus other than a camera for measuring at least one physico-chemical characteristic of the skin; and
a processor for delivering information relating to the skin by processing at least one acquired image and/or a measurement performed by the apparatus.

The term “physico-chemical characteristic” is used to mean the complexion, the sebum concentration, the degree of hydration, or the degree of firmness of the skin.

The invention may render it possible to determine precisely a state of the subject's skin and thus to determine the subject's specific needs, because the skin is analyzed not only on the basis of at least one image thereof suitable for comparison with a reference image, but also on the basis of at least one non-optical measurement.

This makes it possible, for example at a point of sale or in a beauty parlor, to give a consumer accurate advice in choosing a makeup and/or care product that is adapted to that consumer's skin.

The camera preferably includes an objective lens that magnifies more than 40 times, and for example 60 times. Such magnification turns out to be appropriate for processing the image in order to quantify the number of pores or of spots on the skin, for example.

From at least one image of the skin acquired by the camera, the processor is advantageously configured to determine at least one of the following: the number, for example the mean number; the size, for example the mean size; and the locations of pores and/or spots on the skin.

In an embodiment of the invention, the camera includes a polarizer making it possible to observe beneath the surface of the skin.

The system may include an appliance for collecting sebum from the skin, which appliance may comprise, for example, a sebum-collecting medium constituted, for example, by a pellet having a face for application to the skin. The processor is advantageously configured to determine the quantity of sebum collected by processing an image of the collector medium.

In addition to the first camera enabling pores and/or spots of the skin to be observed, the system may further include a second camera having an objective lens with lower magnification, for example with a magnification factor of less than 20, e.g. equal to 10, in order to acquire at least one image of the skin enabling the depth of wrinkles therein to be analyzed.

The processor may be configured, for example, to determine a degree of skin roughness on the basis of an image of wrinkles in the skin acquired using such a camera.

The system may also include an elasticity meter for determining the degree of firmness of the skin, and/or a corneometer for determining the degree of hydration of the skin.

Preferably, the processor is configured to enable data obtained for a subject to be compared with reference data.

The reference data may be obtained for example from measurements taken for example on an individual or a group of individuals considered as having ideal skin, and associated with respective age ranges and with a given population type. The reference skin may or not be an ideal skin.

In order to determine an ideal skin for a given age range and a given population, it may be possible for example to evaluate at least one skin characteristic for each person of this population by giving each person a score selected in a scale. An ideal skin may for instance correspond to the skin having the best score for this population or a skin having a score falling within a predetermined percentile, for example in the best ten percentiles.

For example, if the skin characteristic is a wrinkle severity, the best score and ideal skin may correspond to the less wrinkled skin for the studied population having a given age range.

An ideal skin may be selected as having a score better than the average or median score determined for a given age range, a given population and a given skin characteristic.

An ideal skin may also be the skin of some well known individuals, known for their beauty, for example some models or actors or actress. An ideal skin may also result from a selection by a panel of different skins, the selection being made for example based on esthetic considerations.

In an embodiment of the invention, the system is configured to enable personal information concerning the subject being examined to be entered, said information possibly comprising indications about at least one of the following, for example: the subject's perception of his or her skin state, preoccupations concerning the skin, and habits in beauty matters.

The system may be configured to provide a questionnaire, for example in electronic form, enabling said information to be obtained in response thereto.

The system may be configured to deliver a synthesized graphics representation, for example of histogram type, presenting the data obtained for a subject in comparison with the reference data, with the representation being, for example, in the form of a page displayed by a display device.

In an embodiment of the invention, system includes a data storage space enabling the data obtained for a subject to be stored.

In another of its aspects, the invention provides a method for providing a beauty advice, the method comprising:
acquiring an image of the skin of a subject enabling the pores and/or spots of the skin to be observed, which skin may optionally have or not been previously subjected to a cosmetic and/or care treatment;
measuring a physico-chemical characteristic of the skin using an apparatus other than a camera; and
providing beauty advice on the basis of the acquired image and/or a measurement performed by the apparatus.

The method may comprise displaying the acquired image simultaneously with an image of a reference skin.

Preferably, at least one of the following, or all of the following, is determined:
the number, size, and location of pores in the skin;
the number, size, and location of spots on the skin;
the complexion of the skin;
the wrinkle depth of the skin;
the quantity of sebum in at least one area of the skin;
the degree of hydration of the skin; and
the degree of firmness of the skin.

In an implementation of the invention, the data determined for the subject is compared with reference data associated with a selected age range, which age range may or may not correspond with the age of the subject. This makes it possible, for example, to determine that the subject presents skin characteristics corresponding to a different age range.

The method may comprise, for example, the step of obtaining personal information concerning the subject, said information containing, for example, indications about the subject's perception of his or her own skin state.

In an implementation of the invention, the result of the analysis is compared with the information provided by the subject on the subject's perception of his or her own skin.

The method may include the step of prescribing one or more care products or one or more diet supplements as a function of the results of the analysis and of the information provided by the subject.

The invention also relates to, according to another of its aspects, a method comprising:
at least one of:
   acquiring at least one image of the skin of a subject;
   measuring at least one physico-chemical characteristic of the skin using an apparatus other than a camera;
   enabling the subject to provide information concerning the perception of his or her own skin;
   comparing at least one information resulting from processing the at least one image and/or the at least one measured characteristic and the information provided by the subject.

The invention also relates to according to another of its aspects, a system comprising:
a first camera for acquiring at least one image of the skin at a magnification that enables the pores and/or the spots of the skin to be observed;
a second camera having an objective lens of lower magnification than that of the first camera in order to acquire at least one image of the skin enabling the wrinkles of the skin to be analyzed;
an elasticity meter for determining the degree of firmness of the skin;
a corneometer for determining the degree of hydration of the skin;
an apparatus for collecting sebum on the skin;
a processor for delivering information relating to the skin by processing at least one acquired image and/or a measurement performed by the apparatus.

The invention also relates to, according to another of its aspects, a method comprising:
measuring at least one physico-chemical characteristic of the skin using an apparatus other than a camera;
comparing the at least one measured characteristic with reference data associated with a group of individuals considered as having ideal skin

### BRIEF DESCRIPTION OF THE DRAWINGS

The invention can be better understood on reading the following detailed description of a non-limiting embodiment of the invention, and on examining the accompanying drawings, in which:

FIG. 1 is a diagrammatic and fragmentary view of an analysis system in accordance with the invention;

FIG. 2 is a diagrammatic and fragmentary view of an apparatus for determining the quantity of sebum on an area of the skin;

FIG. 3 is a fragmentary diagram illustrating the use of the FIG. 2 apparatus;

FIG. 4 is a fragmentary diagram illustrating the acquisition of an image of the application face of the sebum collector medium;

FIG. 5 is a flow chart showing the various states in a method for giving advise in beauty matters; and

FIGS. 6 to 18 are diagrammatic and fragmentary views of screen pages displayed by the system of FIG. 1.

### MORE DETAILED DESCRIPTION

FIG. 1 shows a system 1 for analyzing the skin P of a subject, the system comprises a computer 2, a printer 3 connected to the computer 2, and a first camera 4 also connected to the computer 2.

The computer 2 may be a conventional computer comprising a central unit 5, a screen 8, and a user interface including a keyboard 9, and a mouse 10.

The first camera 4 has an objective lens that magnifies sixty times and it is fitted with a polarizer (not shown) of adjustable orientation, and it includes built-in lighting.

The system 1 further comprises, connected to the computer 2:
a second camera 11 having an objective lens with ten times magnification;
an elasticity meter 12; and
a corneometer 13.

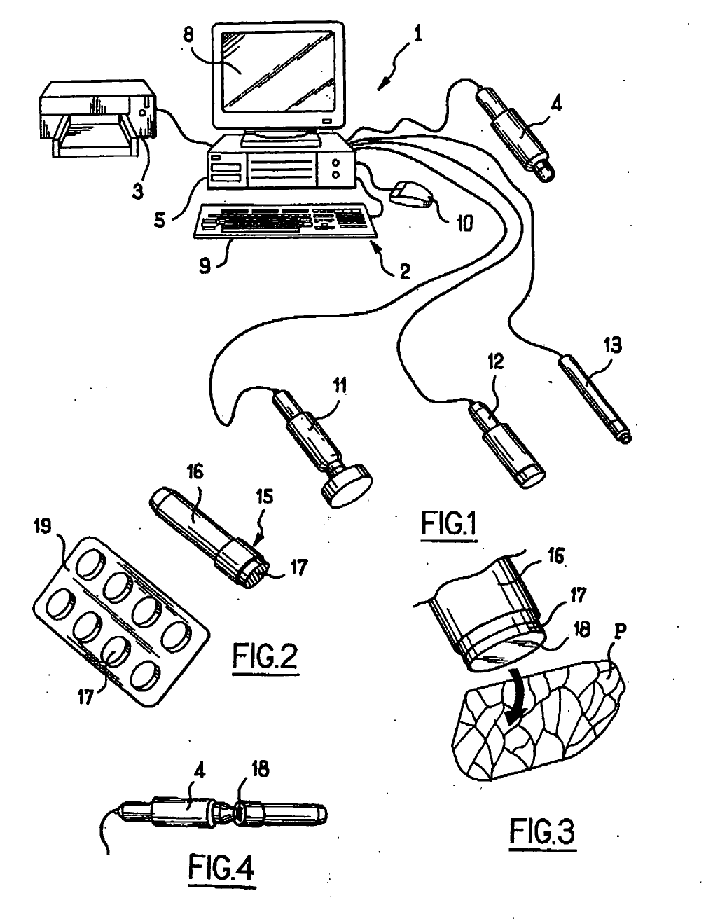

The analysis system 1 may further include an apparatus 15, shown in FIG. 2, for collecting sebum from the skin of a subject.

The apparatus 15 is constituted by a sebumeter, for example.

This apparatus 15 comprises a handle 16 with one end arranged to receive a sebum-collector pellet 17. In the example described, the pellet 17 is packaged prior to use in a strip 19.

The apparatus 15 is used as follows.

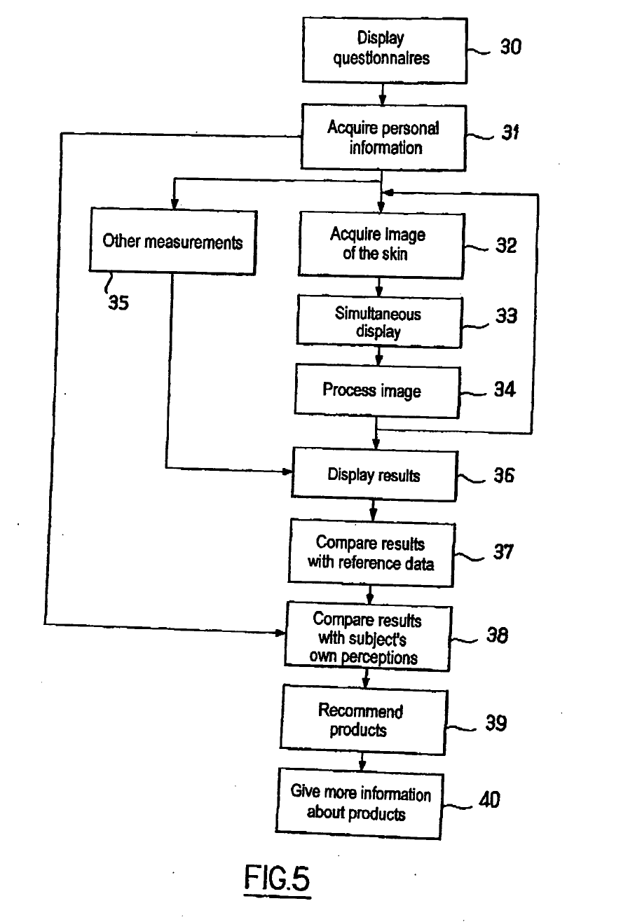

A sebum-collector pellet 17 is fastened to the handle 16 and then a face 18 of the pellet 17 is applied to the skin P of the subject. After removing the pellet, the camera 4, as shown in FIG. 4, is used to acquire an image of the face 18 of the pellet 17 that has collected the sebum.

Processing the image makes it possible to determine the quantity of sebum that has been collected.

In the example described, the system 1 is also arranged to enable personal information concerning the subject to be input.

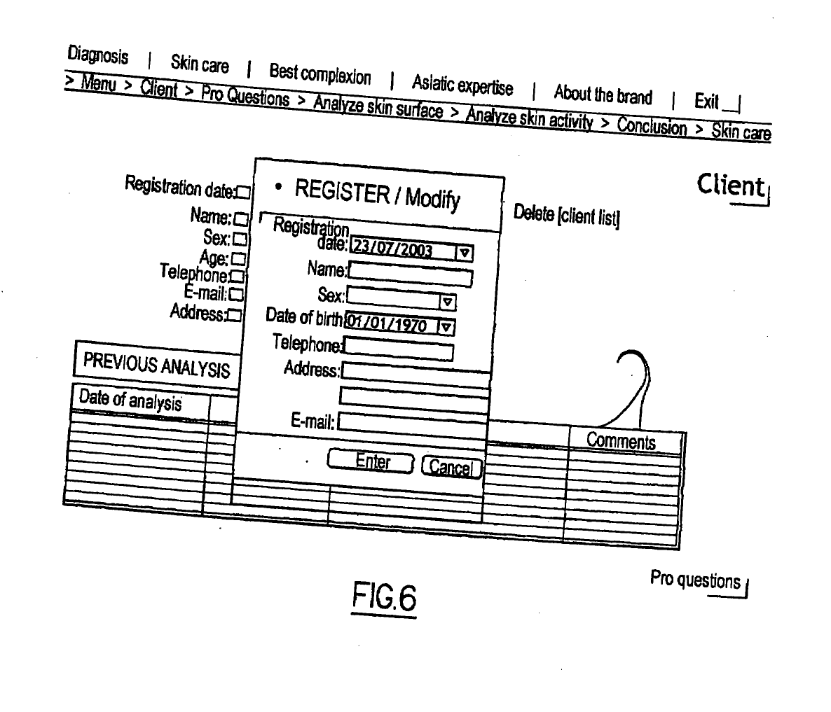

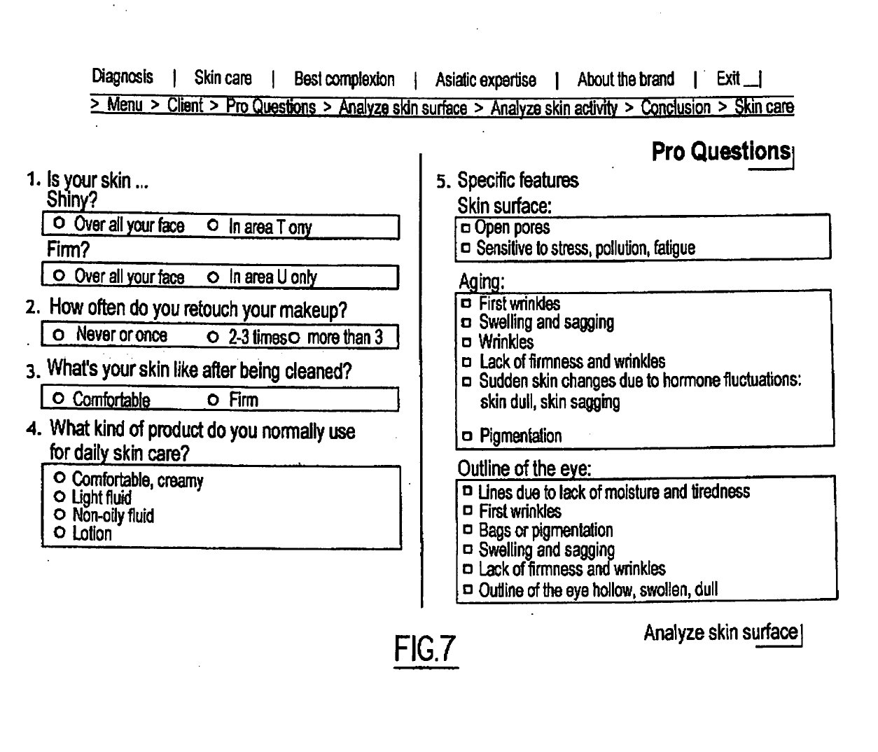

To do this, the computer 2 proceeds, as shown in FIG. 5, in a first step 30 by displaying questionnaires on the screen 8, such as those shown respectively in FIGS. 6 and 7.

The questionnaire shown in FIG. 6 serves during a step 31 to enter information such as date of registration, name, sex, date of birth, telephone number, postal address, and e-mail address of the subject.

This information is stored in the central unit 5, for example.

The questionnaire shown in FIG. 7 comprises various questions concerning the perceptions that subjects have of their own skin, their makeup habits, and their expectations in beauty matters.

By way of example, these questions can be of the following type:
is your skin shiny?
does your skin seem to be firm?
how often do you retouch your makeup: never, or one, two, or three times, more than three times?
after being cleaned, does your skin seem to be: comfortable? firm?

In step 32, a subject or a beautician, as appropriate, proceeds to acquire an image 40 of the skin using the first camera 4.

The magnification of the first camera 4 makes it possible to observe the pores and the spots of the skin.

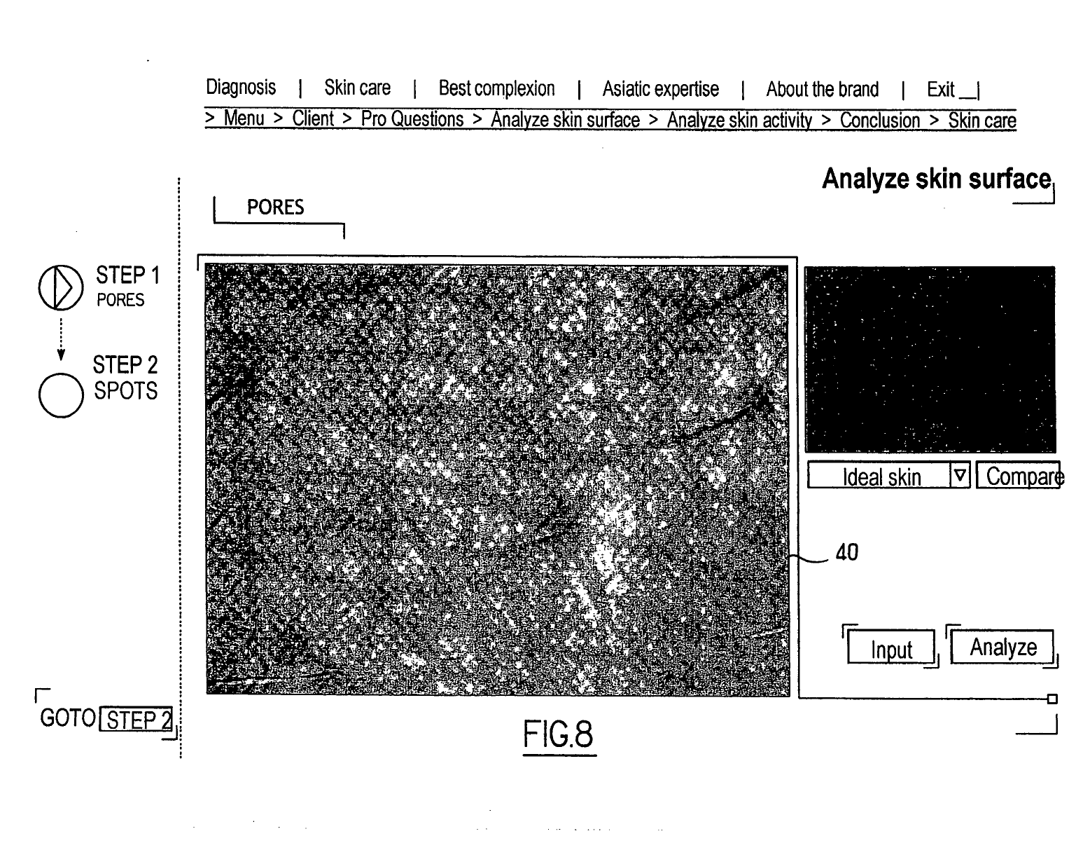

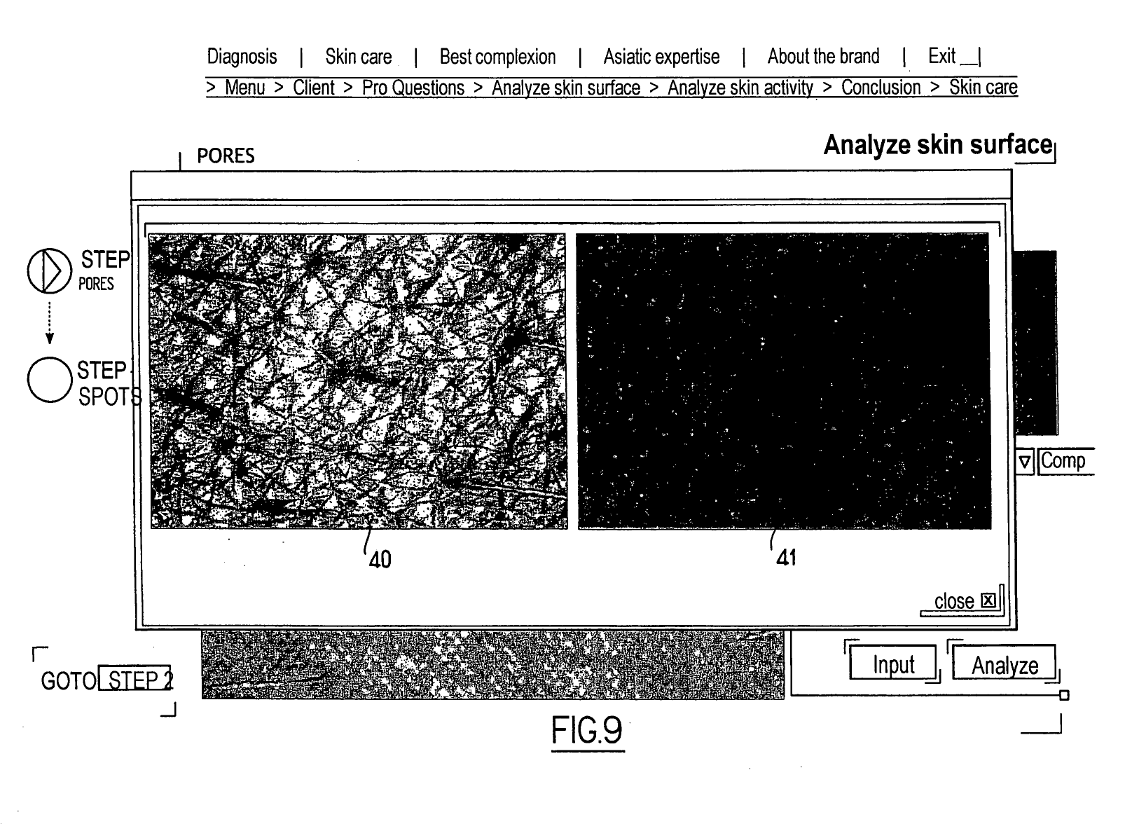

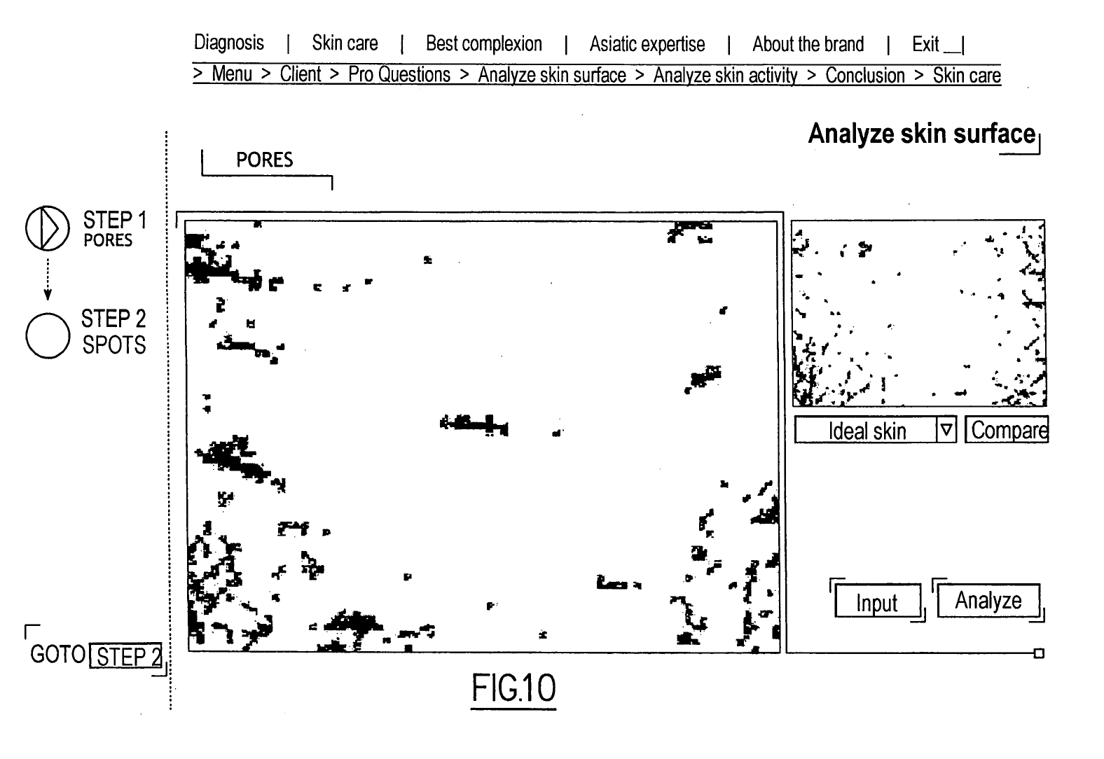

The image 40 is displayed on the screen 8, as can be seen in FIG. 8.

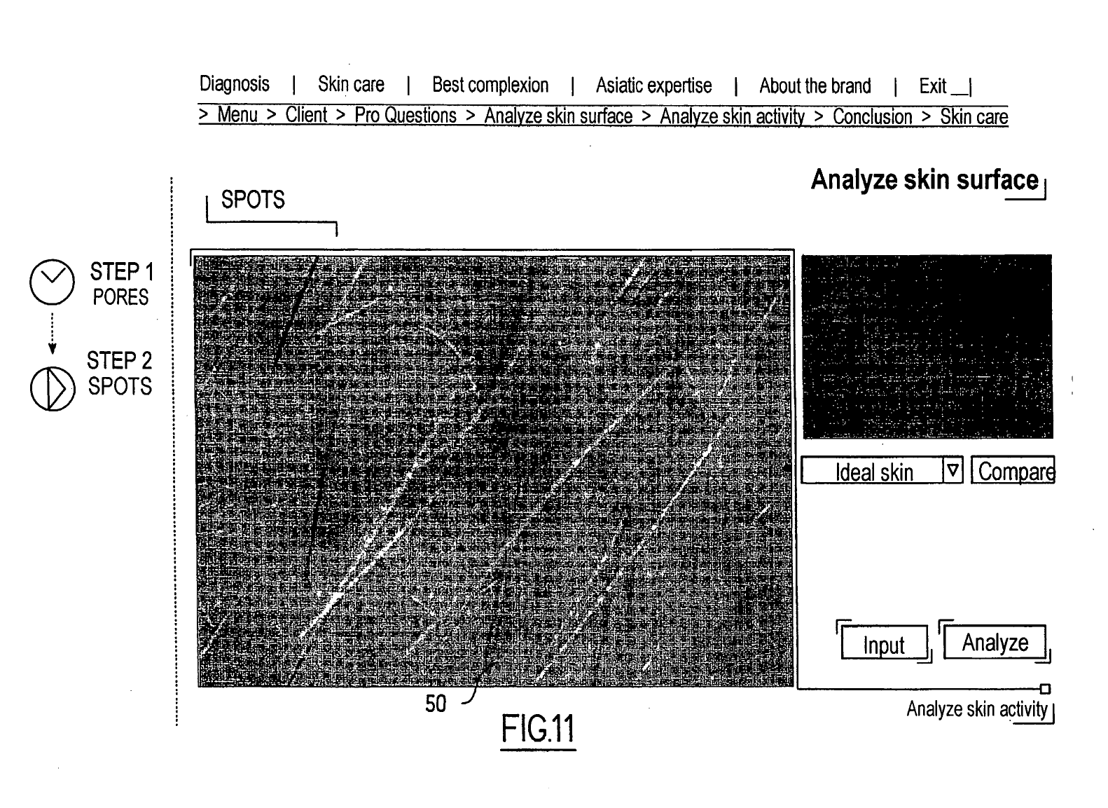

In step 33, the image 40 of the subject's skin is displayed simultaneously with an image 41 of a reference skin, these images having the same format, as shown in FIG. 9.

In the example described, the reference skins are associated with respective age ranges, and each is obtained from the skin of five people in a given age range and belonging to a given population type, the people in question being considered as having ideal skin.

The image of the subject's skin can thus be compared with an image of a reference skin for the subject's age range and for the subject's population type.

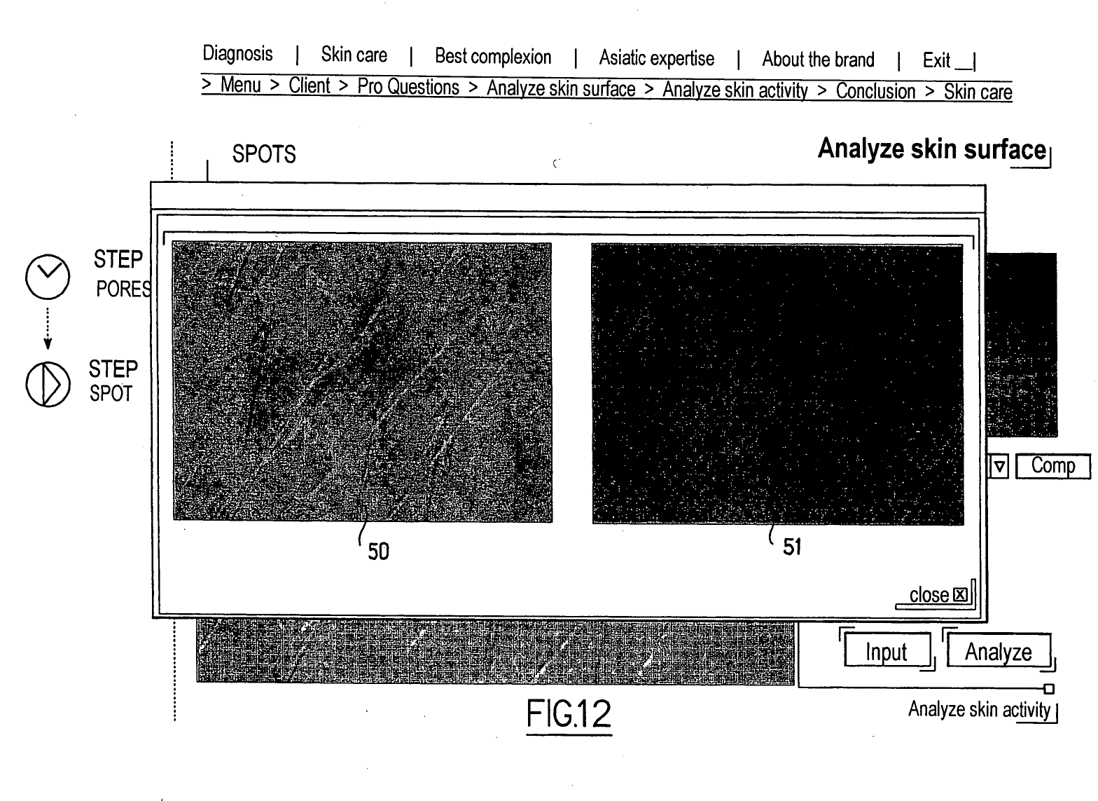

In the following step 34, the image 40 is processed by binary detection, as shown in FIG. 10, in particular for the purpose of determining the number, the size, and the locations of pores in the skin.

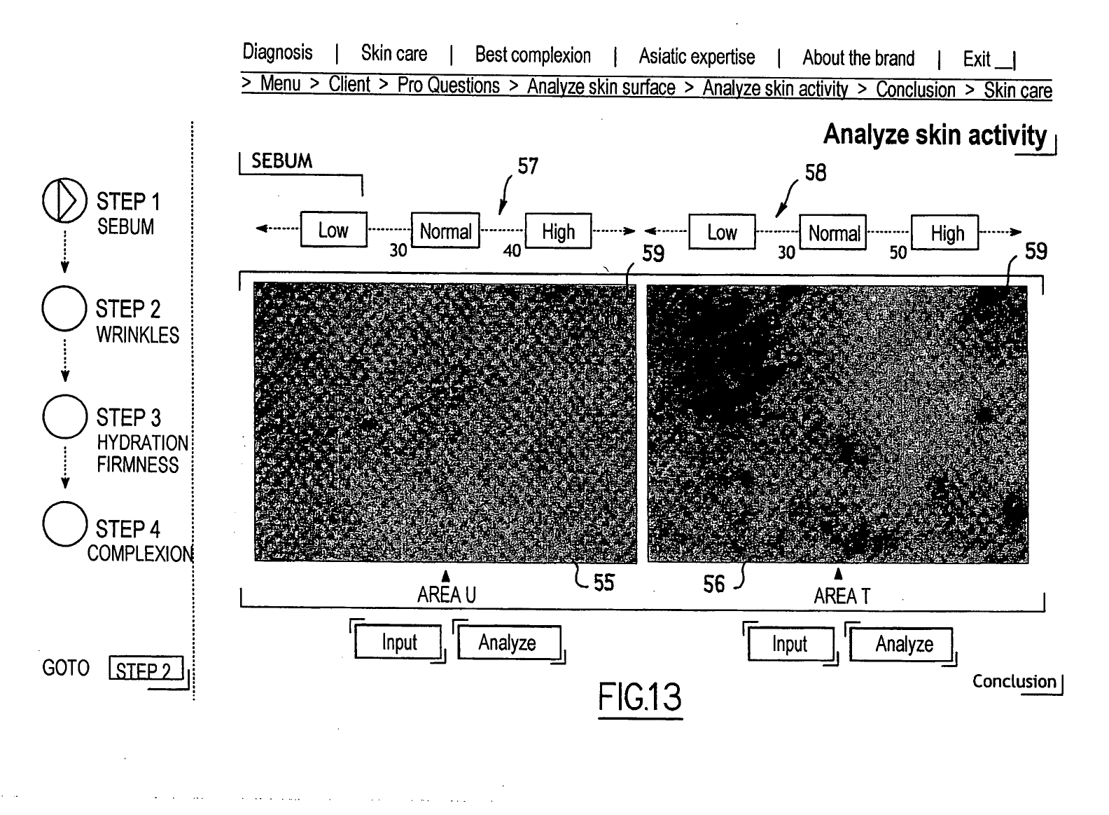

Steps 32 to 34 can be repeated when observing spots on the skin.

In which case, in step 32, an image 50 of the skin as shown in FIG. 11 is acquired by using the camera 4 which is fitted with a polarizer.

In step 33, the image 50 can be displayed simultaneously with an image 51 of a reference skin for comparison purposes, as shown in FIG. 12.

In step 34, using binary detection, it is possible to determine in particular the number, the size, and the locations of spots on the skin.

In order to refine the analysis of the skin, it is possible to proceed with additional measurements of the skin in step 35.

In particular, it is possible to measure the quantity of sebum on the subject's skin.

For this purpose, the apparatus 15 is used that serves to collect sebum on a pellet 17.

Using the camera 4, an image is acquired of the application face of the pellet 17.

This operation is performed respectively for the zone U of the face and the zone T of the face. The zone U corresponds to the region of the face extending from one cheek to the other going via the chin. The zone T corresponds to the region of the face defined by the forehead and the nose.

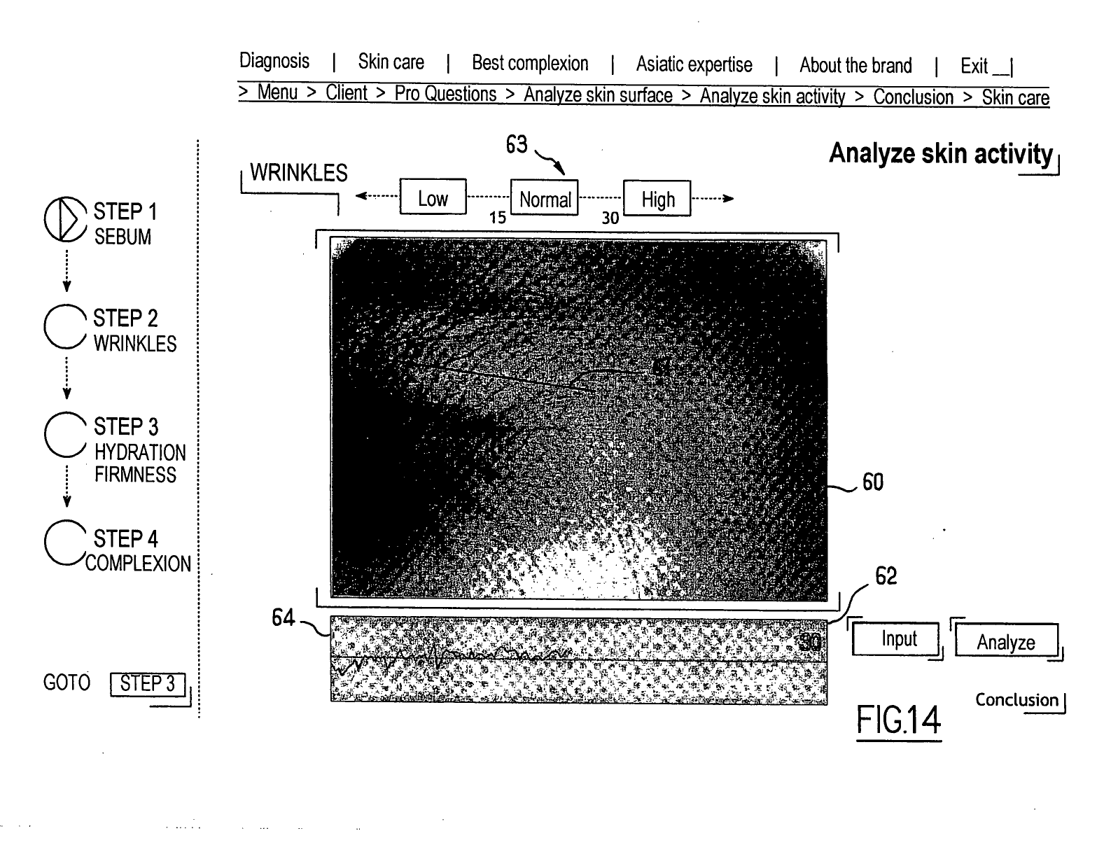

FIG. 13 shows two images 55 and 56 obtained respectively from the zone U and from the zone T. From these images 55 and 56, the central unit 5 calculates the quantity of sebum for each zone.

The values obtained 59 are compared with respective reference values, e.g. presented on scales 57 and 58, thus making it possible to determine whether the presence of sebum is low, normal, or high.

For example, in the zone U, the measured value corresponds to a situation where sebum is low. In contrast, the value measured in the zone T corresponds to a situation in which the quantity of sebum is in excess.

In order to determine the roughness of the skin, an image 60 is acquired using the camera 11.

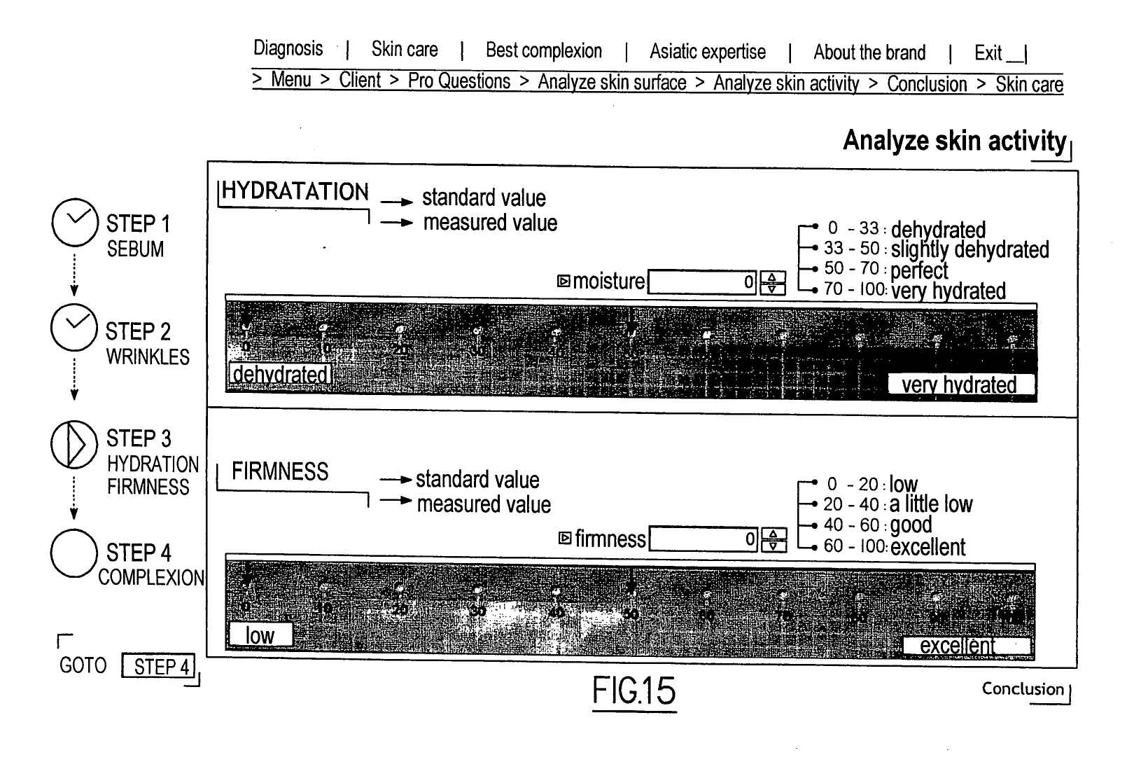

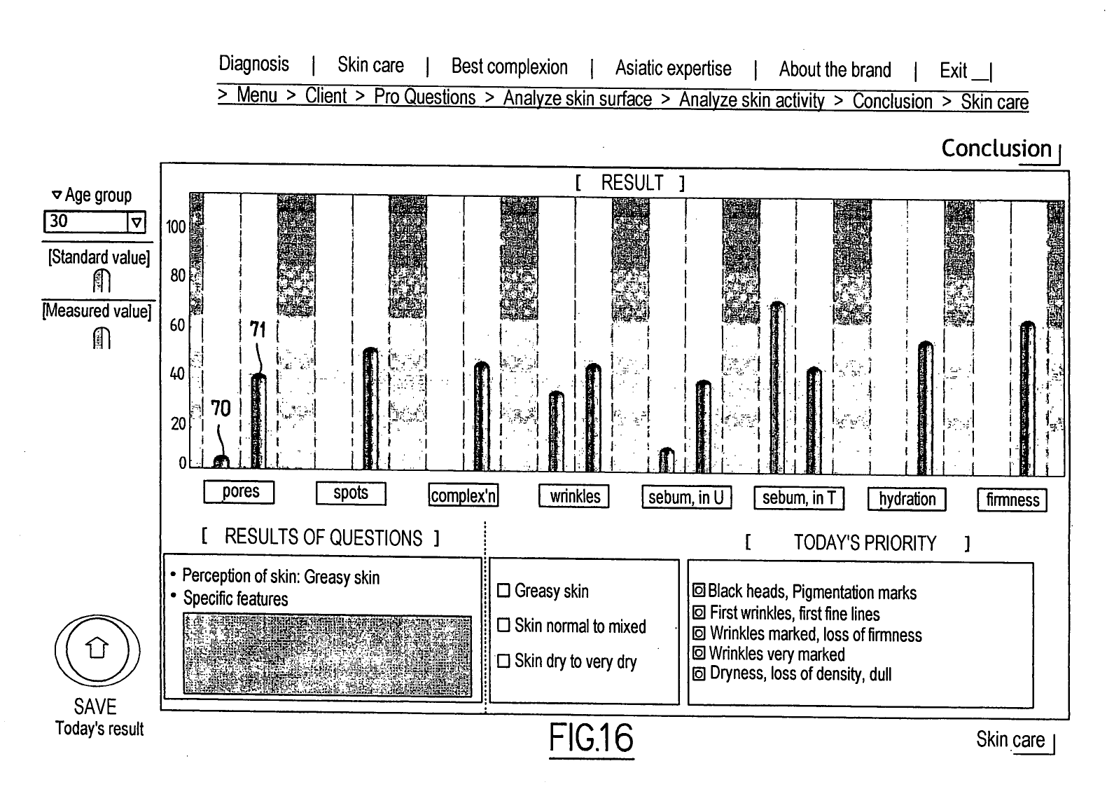

The acquired image 60 is displayed, as shown in FIG. 14, and reveals, for example, the wrinkles around the eye.

The central unit 5 is arranged to determine from said image 60 and from a selected line 61 a degree of roughness 62 for the skin by evaluating variations in the relief of the skin along said line 61, as represented by the graph 64.

This measured degree may be compared with reference values 63 that are displayed above the image 60.

As can be seen, the degree measured in the example described corresponds to a normal situation.

Thereafter, the degree of firmness of the skin is determined by means of an elasticity meter 12 and the degree of hydration of the skin is determined using the corneometer 13.

The measured values for hydration and firmness can be compared respectively with hydration and firmness scales, as shown in FIG. 14.

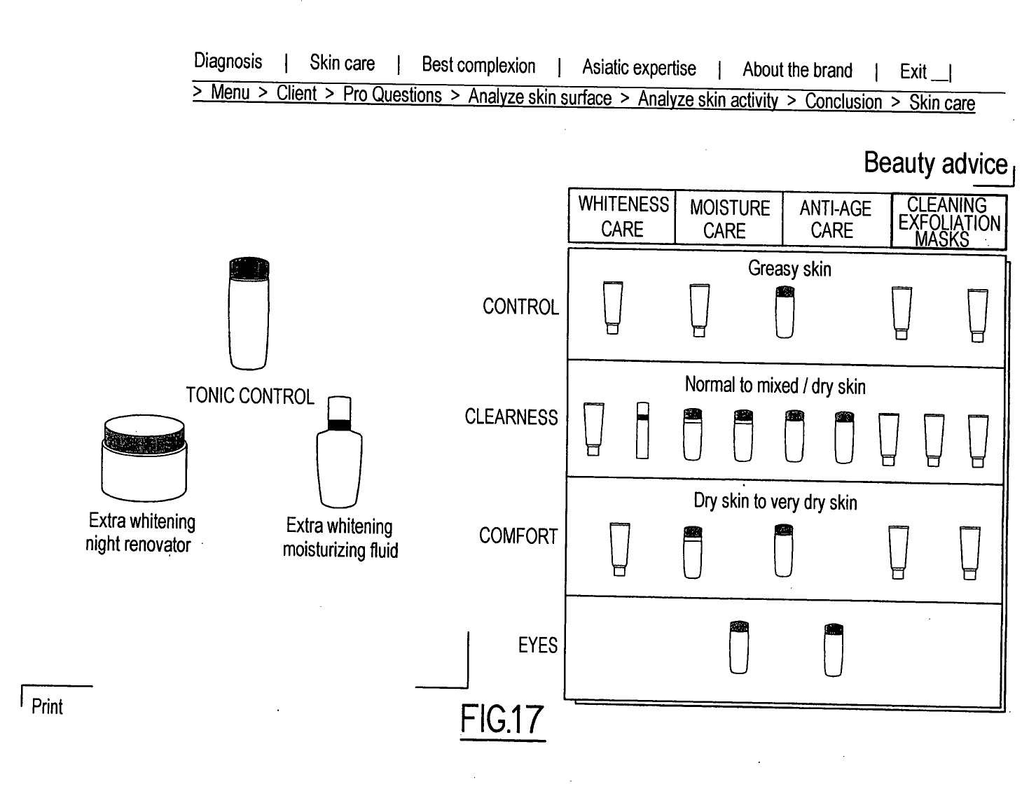

After taking these measurements, in a step 36, the results obtained relative to the reference values are displayed together in the form of a histogram type graphical representation, as can be seen in FIG. 16.

This gives an overall view of the results.

The graphical representation of the results can be printed on paper using the printer 3 or can be stored in the central unit 5, for example.

These results can also be sent over a telephone or computer network, in particular over the Internet, for example to the portable terminal or the personal computer of the subject.

Columns 70 correspond to the values as measured for the skin of the subject and they are placed beside columns 71 corresponding to reference values respectively for each characteristic of the skin, namely, for example: numbers of pores, of spots, complexion, wrinkles, quantity of sebum, respectively in zone U and in zone T, hydration, and firmness.

The reference values are associated with respective age ranges and are obtained, like the reference skin images, from measurements on groups of individuals considered as having ideal skin.

In step 37, the results of the analysis are compared with reference data, in particular data associated with a particular age range, in order to determine the defects and the qualities of the skin of the subject.

For example, the beautician may conclude that a subject who is 40 years old has skin hydration corresponding to that of a 30-year old.

Conversely, the beautician might find that for a 30-year old subject, skin firmness corresponds to that of a 40-year old person.

Under the histogram, there can be seen the information given by the subject concerning the subject's own perception of his or her skin, and on the right there can be seen the conclusions deduced from the measurements, enabling them to be compared in step 38.

On sight firstly of the information relating to the subject's own perception of his or her skin and secondly of the results of the measurements, the beautician can, in a step 39, give advice to the consumer in choosing makeup, care, or treatment products, and possibly also diet supplements.

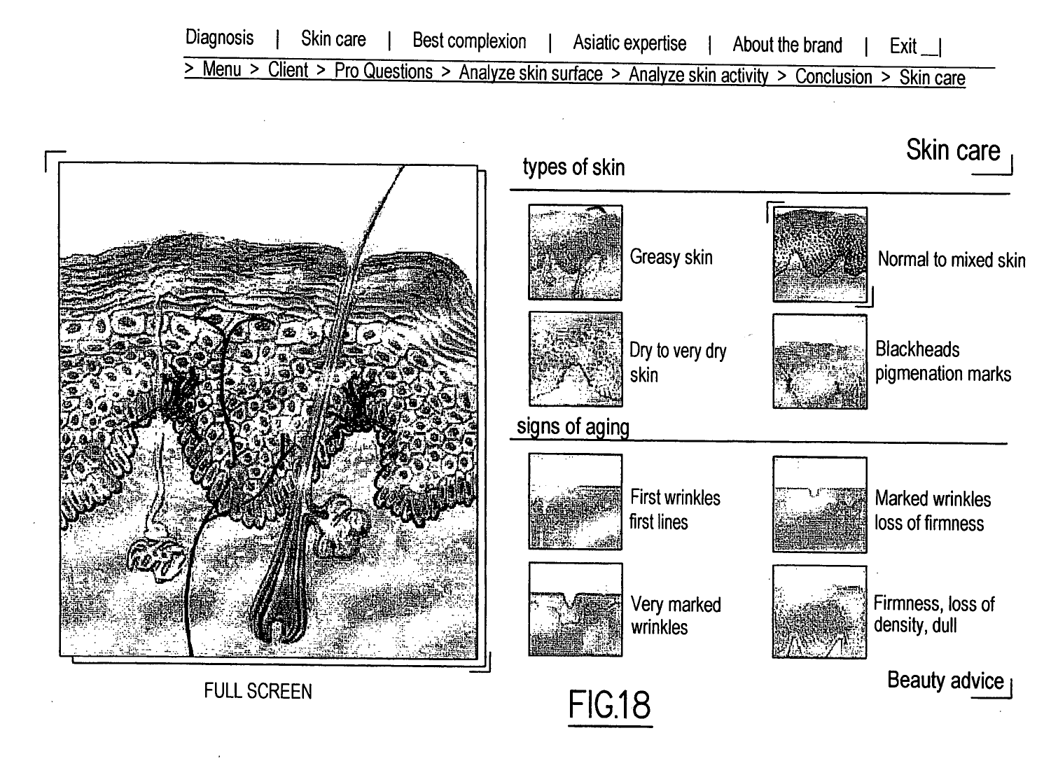

As can be seen in FIG. 17, it is possible to recommend those cosmetic products in a set of cosmetics that are most suitable for the subject.

Finally, using screen pages such as the page shown in FIG. 18, the beautician can give the subject explanations concerning the state of his or her skin.

Throughout the description, including in the claims, the term “comprising a” should be understood as being synonymous with “comprising at least one” unless specified to the contrary.

The invention is not limited to the example described above.

In particular, the analysis system may include other measurement apparatuses for evaluating skin state.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A system for analyzing the skin of a subject, the system comprising:
a camera for acquiring at least one image of the skin at a magnification that enables the pores and/or the spots of the skin to be observed;
a display device for displaying simultaneously at least one acquired image and an image of a reference skin;
an apparatus other than a camera for measuring at least one physico-chemical characteristic of the skin; and
a processor for delivering information relating to the skin by processing at least one acquired image and/or a measurement performed by the apparatus.

2. A system according to claim 1, wherein the camera has an objective lens with at least 40 times magnification.

3. A system according to claim 1, wherein the camera has an objective lens with substantially 60 times magnification.

4. A system according to claim 1, wherein the processor is configured to determine from at least one image of the skin acquired by the camera at least one of the following: the number, the size, and the locations of pores in the skin.

5. A system according to claim 1, wherein the processor is configured to determine, from at least one image of the skin acquired by the camera, at least one of the following: the number, the size, and the locations of spots on the skin.

6. A system according to claim 1, wherein the camera comprises at least one polarizer.

7. A system according to claim 1, comprising at least one apparatus for collecting sebum on the skin.

8. A system according to claim 7, wherein the apparatus comprises at least one sebum-collector medium comprising a pellet having a face for application against the skin.

9. A system according to claim 8, wherein the processor is configured to determine the quantity of collected sebum by processing at least one image of the collector medium.

10. A system according to claim 1, comprising a second camera having an objective lens of lower magnification than that of the first camera, in order to acquire at least one image of the skin enabling the wrinkles of the skin to be analyzed.

11. A system according to claim 10, wherein the lower magnification factor is less than 20 times.

12. A system according to claim 10, wherein the lower magnification factor is substantially equal to 10.

13. A system according to claim 10, wherein the processor is configured to determine the degree of roughness of the skin from at least one image of wrinkles of the skin.

14. A system according to claim 1, comprising an elasticity meter for determining the degree of firmness of the skin.

15. A system according to claim 1, comprising a corneometer for determining the degree of hydration of the skin.

16. A system according to claim 1, wherein the processor is configured to enable the data that results from the analysis to be compared with reference data.

17. A system according to claim 16, wherein the reference data is obtained from measurements taken on a group of individuals considered as having ideal skin, the reference data being associated with respective age ranges and with a given population type.

18. A system according to claim 1, the system being configured to enable personal information about the subject to be input.

19. A system according to claim 18, the system being configured to provide at least one questionnaire enabling said information to be obtained in reply.

20. A system according to claim 19, wherein the system is configured to provide electronically the at least one questionnaire.

21. A system according to claim 1, the system being configured to deliver a graphical representation showing the results of the analysis in comparison with the reference data.

22. A system according to claim 21, wherein the graphical representation comprises a histogram.

23. A system according to claim 21, wherein said results are shown on a page that is displayed by the display device.

24. A system according to claim 1, comprising data storage space for storing the results of the analysis.

25. A system according to claim 1, configured for delivering data via a network.

26. A system according to claim 25, wherein the network is a telephone network.

27. A system according to claim 25, wherein the network is a computer network.

28. A system according to claim 25, configured for sending data to at least one of a portable terminal and a personal computer of the subject.

29. A method for providing a beauty advice, the method comprising:
acquiring at least one image of the skin of a subject enabling the pores and/or spots of the skin to be observed,
measuring at least one physico-chemical characteristic of the skin using an apparatus other than a camera; and
providing a beauty advice on the basis of the acquired image and/or a measurement performed by the apparatus.

30. A method according to claim 29, comprising:
displaying the acquired image simultaneously with an image of a reference skin.

31. A method according to claim 29, comprising:
determining at least one of the following:
the number, size, and locations of spots on the skin;
the complexion of the skin;
the wrinkle depth of the skin;
the quantity of sebum in at least one area of the skin;
the degree of hydration of the skin; and
the degree of firmness of the skin.

32. A method according to claim 29, comprising:
determining all of the following:
the number, size, and locations of spots on the skin;
the complexion of the skin;
the wrinkle depth of the skin;
the quantity of sebum in at least one area of the skin;
the degree of hydration of the skin; and
the degree of firmness of the skin.

33. A method according to claim 29, comprising:
determining the quantity of sebum in at least one of the zone U and the zone T of the face.

34. A method according to claim 29, comprising:
comparing the data determined for the subject with reference data associated with a selected age range.

35. A method according to claim 34, comprising:
determining at least one of the defects and the qualities of the skin of the subject.

36. A method according to claim 29, comprising:
obtaining personal information concerning the subject.

37. A method according to claim 29, comprising:
comparing a result of processing the acquired image and/or a measurement performed by the apparatus with information provided by the subject on the subject's perception of his or her own skin.

38. A method according to claim 29 comprising:
prescribing one ore more care products or one more diet supplements as a function of the results of the analysis and of information provided by the subject.

39. A method comprising:
at least one of:
acquiring at least one image of the skin of a subject;
measuring at least one physico-chemical characteristic of the skin using an apparatus other than a camera;
enabling the subject to provide information concerning the perception of his or her own skin;
comparing at least one information resulting from processing the at least one image and/or the at least one measured characteristic and the information provided by the subject.

40. A method according to claim 39, wherein a beauty advice is provided based on said comparison.

41. A method according to claim 39, wherein a recommendation of a product is provided based on said comparison.

42. A system for analyzing the skin of a subject, the system comprising:
a first camera for acquiring at least one image of the skin at a magnification that enables the pores and/or the spots of the skin to be observed;
a second camera having an objective lens of lower magnification than that of the first camera in order to acquire at least one image of the skin enabling the wrinkles of the skin to be analyzed;
an elasticity meter for determining the degree of firmness of the skin;
a corneometer for determining the degree of hydration of the skin;
an apparatus for collecting sebum on the skin;
a processor for delivering information relating to the skin by processing at least one acquired image and/or a measurement performed by the apparatus.

43. A method comprising:
measuring at least one physico-chemical characteristic of the skin using an apparatus other than a camera;
comparing the at least one measured characteristic with reference data associated with a least one individual considered as having an ideal skin.

44. A method according to claim 43, wherein the reference data is associated with respective age ranges and a given population type.

45. A method according to claim 43, comprising:
determining at least one of the defects and the qualities of the skin of the subject.

46. A method according to claim 43, comprising:
determining at least one of the following:
the number, size, and locations of spots on the skin;
the complexion of the skin;
the wrinkle depth of the skin;
the quantity of sebum in at least one area of the skin;
the degree of hydration of the skin; and
the degree of firmness of the skin.

47. A method according to claim 43, wherein a beauty advice is provided based on said comparison.

48. A method according to claim 43, wherein a recommendation of a product is provided based on said composition.
